(19) United States
(12) Patent Application Publication
(10) Pub. No.: US 2021/0096125 A1
(43) Pub. Date: Apr. 1, 2021

(54) CELL-CELL INTERACTION ANALYSIS VIA DROPLET MICROFLUIDICS

(71) Applicant: Scribe Biosciences, San Francisco, CA (US)

(72) Inventors: Maithreyan Srinivasan, San Francisco, CA (US); Russell Cole, San Francisco, CA (US)

(21) Appl. No.: 17/032,922

(22) Filed: Sep. 25, 2020

Related U.S. Application Data
(60) Provisional application No. 62/907,334, filed on Sep. 27, 2019.

(51) Int. Cl. G01N 33/50; C12Q 1/6869; B01L 3/00

(52) U.S. Cl. CPC G01N 33/5005; B01L 3/502792; C12Q 1/6869

(57) ABSTRACT

The present invention provides systems, kits, and methods for analyzing cell-cell interactions, such as transmembrane proteins binding to surface displayed variable regions, via discrete entity (e.g., droplet) microfluidics. In certain embodiments, a plurality of first discrete entities and a plurality of second discrete entities are merged on a substrate to generate a plurality of merged fixed entities (e.g., fixed via an electrical force), each of which contains one cell expressing a transmembrane (TM) protein and labeled clonal cells displaying a heterologous antibody variable region. In certain embodiments, any binding of the clonal cells to the TM expressing cell is detected in each merged fixed entity, and the clonal cells found to bind are treated in order to sequence the nucleic acid encoding the variable region.

The present application claims priority to U.S. Provisional application Ser. No. 62/907,334 filed Sep. 27, 2019, which is herein incorporated by reference.

### FIELD OF THE INVENTION

The present invention provides systems, kits, and methods for analyzing cell-cell interactions, such as transmembrane proteins binding to surface displayed variable regions, via discrete entity (e.g., droplet) microfluidics. In certain embodiments, a plurality of first discrete entities and a plurality of second discrete entities are merged on a substrate to generate a plurality of merged fixed entities (e.g., fixed via an electrical force), each of which contains one cell expressing a transmembrane (TM) protein and labeled clonal cells displaying a heterologous antibody variable region. In certain embodiments, binding of the clonal cells to the TM expressing cell is detected in each merged fixed entity, and the clonal cells found to bind are treated in order to sequence the nucleic acid encoding the variable region.

### BACKGROUND

It is estimated that there are about 20,000 protein coding genes in the human genome, about 3000 of which are considered appropriate drug candidates and more than 2300 of the 3000 have yet to be targeted by therapies (Rodgers et al., Nat. Rev. Drug Discov., vol. 17, p. 301, January 2018). A 2013 NIH study identified two reasons for the lack of progress: lack of consolidated information on the druggable genome and lack of high throughput technologies to functionally characterize the 2300 targets (Rodgers et al.). Based on the findings of the 2013 study, NIH initiated a program called Illuminating the Druggable Genome (IDG). One of the findings of the program was to focus research on non-olfactory GPCRs, ion channels and protein kinases to discover therapies. Abnormal activities of GPCRs are linked to various human diseases and indeed, approximately 50% of approved drugs act on signal transduction systems involving GPCRs, 30% of which act on GPCRs directly (Xiao et al., Combinatorial Chemistry & High Throughput Screening, vol. 11, no. 3. pp. 195-215, 2008). Out of 400 non-olfactory GPCRs that are druggable, about 150 GPCRs have either small molecule or peptide drugs. Small molecule and peptide drugs depend on the extent of extracellular domain exposure and frequently result in liver toxicities. So, antibody therapies are being sought as an alternative.

Identifying antibodies for transmembrane proteins such as G-protein coupled receptors (GPCRs), ion channels, transporters etc., are challenging because transmembrane (TM) proteins are difficult to express in heterologous systems in sufficient quantities to immunize animals. In addition, retaining the native conformation and function of TM proteins in heterologous systems can also pose significant challenges. Furthermore, immunizing animals and characterizing antibodies takes months and is an inefficient process[3] [4].

Existing methods to identify antibodies for GPCRs (and other TM proteins) include traditional methods of immunizing animals and identifying the B-cell clones with highest affinity. However, this method is time consuming and is not suitable for screening multiple targets and hundreds and thousands of antibody variants. Other methods such as phage display using a synthetic library where antibodies expressed on the phage coat is screened using purified antigen (GPCR) and repeated bind/wash steps enrich for strong binders (panning). However, synthetic libraries are expensive and available only from select commercial providers, thereby limiting their broad utilization. While phage-display techniques enable isolation of high-affinity binders, identification of functional clones that recognize a defined conformation remains challenging, despite select successes. Thus, technologies that can identify antibodies in functional assays are essential for successful lead generation.

### SUMMARY OF THE INVENTION

The present invention provides systems, kits, and methods for analyzing cell-cell interactions, such as transmembrane proteins binding to surface displayed variable regions, via discrete entity (e.g., droplet) microfluidics. In certain embodiments, a plurality of first discrete entities and a plurality of second discrete entities are merged on a substrate to generate a plurality of merged fixed entities (e.g., fixed via an electrical force), each of which contains one cell expressing a transmembrane (TM) protein and labeled clonal cells displaying a heterologous antibody variable region. In certain embodiments, any binding of the clonal cells to the TM expressing cell is detected in each merged fixed entity, and the clonal cells found to bind are treated in order to sequence the nucleic acid encoding the variable region.

In some embodiments, provided herein are methods comprising on or more or each of the steps of: a) flowing a plurality of discrete entities through a micro-fluidic device in a carrier fluid, wherein the discrete entities are insoluble and/or immiscible in the carrier fluid, and wherein each of the second discrete entities comprises: i) one, and no more than one, transmembrane protein expressing cell (TM cell), or ii) a plurality of TM cells where each of said TM cells expresses the same transmembrane protein; b) directing the carrier fluid and the plurality of discrete entities through a delivery orifice to a substrate such that each of the discrete entities merges with one of a plurality of affixed entities present on the substrate thereby generating a plurality of merged affixed entities, wherein each of the affixed entities comprises a plurality of clonal surface display cells (SD cells), wherein each of the clonal SD cells comprises: i) an outer surface displaying a polypeptide, wherein the polypeptide comprises at least one heterologous antibody variable region that is unique among the heterologous variable regions present in the plurality of affixed entities, ii) a nucleic acid sequence encoding the polypeptide, and iii) a detectable protein; and c) detecting (directly or indirectly), in each of the plurality of merged affixed entities, whether the clonal SD cells bind to the TM cell or to the plurality of TM cells.

In other embodiments, provided herein are methods comprising one or more or each or each of the steps of: a) encapsulating a plurality of surface display cells (SD cells) into a plurality of first discrete entities such that each of the first discrete entities comprises one, and no more than one, of the SD cells, and wherein each of the SD cells comprises: i) an outer surface displaying a polypeptide, wherein the polypeptide comprises at least one heterologous antibody variable region that is unique among the heterologous variable regions from the plurality of SD cells, ii) a nucleic acid sequence encoding the polypeptide, and iii) a detectable protein; b) flowing the plurality of first discrete entities through a micro-fluidic device in a carrier fluid, wherein the first discrete entities are insoluble and/or immiscible in the carrier fluid; c) directing the carrier fluid and the plurality of first discrete entities through a delivery orifice to a substrate such that each of the first discrete entities are individually affixed to the substrate thereby generating a plurality of affixed entities; d) incubating the plurality of affixed entities such that each of the SD cells divides at least once, thereby generating clonal SD cells in each of the affixed entities; e) flowing a plurality of second discrete entities through a micro-fluidic device in a carrier fluid, wherein second discrete entities are insoluble and/or immiscible in the carrier fluid, and wherein each of the second discrete entities comprises: i) one, and no more than one, TM cell expressing at least one transmembrane protein, or ii) a plurality of TM cells where each of said TM cells expresses the same transmembrane protein; f) directing the carrier fluid and the plurality of second discrete entities through a delivery orifice to the substrate such that each of the second discrete entities merges with one of the affixed entities thereby generating a plurality of merged affixed entities; and g) detecting, in each of the plurality of merged affixed entities, directly or indirectly whether the clonal SD cells bind to the TM cell or to the plurality of TM cells.

In particular embodiments, the methods further comprise: identifying at least one merged affixed entity where the multiple SD cells bound to the TM cell, or plurality of TM cells, and adding a composition to the at least one merged affixed entity, wherein the composition comprises barcoded nucleic acid sequences that bind to the nucleic acid sequence encoding the polypeptide. In other embodiments, the methods further comprise: i) sequencing the nucleic acid sequence encoding the polypeptide to determine the sequence encoding the heterologous antibody variable region. In further embodiments, the detecting comprises detecting the position of the SD cells via the detectable protein.

In some embodiments, provided here are systems comprising: a) a substrate comprising a first surface; b) a layer of fluid covering at least part of the first surface; and c) a plurality of affixed entities, wherein each of the affixed entities are independently affixed to the substrate under the layer of fluid, wherein the plurality of affixed entities are insoluble and/or immiscible in the layer of fluid, wherein each of the plurality of affixed entities comprises: i) one, and no more than one, TM cell expressing at least one transmembrane protein, or a plurality of TM cells where each of said TM cells expresses the same transmembrane protein, and ii) multiple identical surface display cells (SD cells), wherein each of the SD cells comprises: A) an outer surface displaying a polypeptide, wherein the polypeptide comprises at least one heterologous antibody variable region that is unique among the heterologous variable regions present in the plurality of affixed entities, B) a nucleic acid sequence encoding the polypeptide, and C) a detectable protein.

In certain embodiments, provided here are systems comprising: a) a substrate comprising a first surface; b) a plurality of microwells; and c) a plurality of affixed entities, wherein one affixed entity is present in each of said microwells, and wherein a layer of humidified air covers said plurality of affixed entities, and wherein each of said plurality of affixed entities comprises: i) one, and no more than one, TM cell expressing at least one transmembrane protein, or a plurality of TM cells where each of said TM cells expresses the same transmembrane protein, and ii) multiple identical surface display cells (SD cells), wherein each of said SD cells comprises: A) an outer surface displaying a polypeptide, wherein said polypeptide comprises at least one heterologous antibody variable region that is unique among the heterologous variable regions present in said plurality of affixed entities, B) a nucleic acid sequence encoding said polypeptide, and C) a detectable protein.

In some embodiments, the detectable protein comprises a fluorescent protein. In other embodiments, the plurality of first discrete entities comprises at least 100 first discrete entities or 1000 first discrete entities or at least 10,000 first discrete entities (e.g., 100 . . . 500 . . . 1000 . . . 2000 . . . 5000 . . . 10, 000 . . . or 20,000). In certain embodiments, the plurality of SD cells comprises at least 100 SD cells or 1000 SD cells or at least 10,000 SD cells (e.g., 100 . . . 500 . . . 1000 . . . 2000 . . . 5000 . . . 10, 000 . . . or 20,000).

In particular embodiments, the substrate is configured to move to different positions under the delivery orifice (e.g., the substrate is a plat panel that is motorized). In other embodiments, the plurality of affixed entities are affixed to the substrate via a force, wherein the force is selected from: gravitational force, electrical force, magnetic force, and combinations thereof. In other embodiments, the plurality of affixed entities are affixed to the substrate via an electrical force. In particular embodiments, the electrical force is a dielectrophoretic force.

In certain embodiments, the discrete entities are droplets (e.g., aqueous droplets). In other embodiments, the droplets comprise an aqueous fluid which is immiscible in the carrier fluid. In further embodiments, the substrate comprises on a first surface a layer of fluid which is miscible with the carrier fluid and immiscible with the aqueous fluid, and wherein the first discrete entities are affixed to the first surface of the substrate following introduction into the layer of fluid on the first surface of the substrate. In additional embodiments, the carrier fluid is an aqueous fluid and the droplets comprise a fluid which is immiscible with the carrier fluid. In additional embodiments, the substrate comprises on a first surface a layer of aqueous fluid which is miscible with the carrier fluid and immiscible with the fluid comprised by the droplets, and wherein the first discrete entities are affixed to the first surface of the substrate following introduction into the layer of aqueous fluid on the first surface of the substrate. In certain embodiments, the first discrete entities are affixed to the substrate via interfacial tension.

In particular embodiments, the polypeptide comprises at least two heterologous antibody variable regions. In other embodiments, the polypeptide comprises at least one of the following: a Fab′, F(ab)2, Fab, a single-chain variable fragment (ScFv), an antibody, a bi-specific antibody, and a minibody. In some embodiments, the nucleic acid sequence comprises an expression vector sequence and a polypeptide encoding sequence. In other embodiments, the nucleic acid sequence further comprises a sequence encoding the detectable protein. In particular embodiments, the nucleic acid sequence further comprises a sequence encoding a linker peptide.

In certain embodiments, the carrier fluid comprises oil. In additional embodiments, the substrate is at least partially covered in oil. In some embodiments, the discrete entities have a diameter of from about 1 to 1000 μm (e.g., 1 . . . 10 . . . 100 . . . 50 . . . or 1000 um).

In certain embodiments, the at least one transmembrane protein comprises a G-protein coupled receptor (GPCR). In further embodiments, the at least one transmembrane protein is over-expressed in the TM cell, or plurality of TM cells (e.g., a mammalian cell line designed to over-express a particular transmembrane protein, such a GPCR). In certain embodiments, the at least one transmembrane protein is heterologous to the TM cell or the plurality of TM cells.

In particular embodiments, the detectable protein produces a detectable signal. In further embodiments, the detecting comprises quantitating the signal. In particular embodiments, the signal produces a sharper peak when the SD cells bind to the TM cell (or plurality of TM cells) in a merged affixed entity, and produces a more diffuse signal when the SD cells do not bind to the TM cell (or plurality of TM cells) in a merged affixed entity. In further embodiments, the discrete entities have a volume of from about 1 femtoliter to about 1000 nanoliters, or from 10 to 800 picoliters. In additional embodiments, the microfluidic device comprises a sorter, and wherein the method comprises sorting, via the sorter, the plurality discrete entities to be delivered through the delivery orifice to the substrate. In other embodiments, the sorter comprises a flow channel comprising a gapped divider comprising a separating wall which extends less than the complete height of the flow channel. In particular embodiments, the plurality of discrete entities are optically scanned prior to the sorting. In certain embodiments, the sorter comprises an optical fiber configured to apply excitation energy to the plurality of discrete entities. In other embodiments, the sorter comprises a second optical fiber configured to collect a signal produced by the application of excitation energy to the plurality of discrete entities. In other embodiments, the optical fiber is configured to apply excitation energy the plurality of discrete entities and collect a signal produced by the application of the excitation energy. In certain embodiments, the sorting is based on results obtained from the optical scan. In some embodiments, the sorter is an active sorter or a passive sorter. In further embodiments, the sorting comprises sorting via dielectrophoresis.

In particular embodiments, the sorter comprises one or more microfluidic valves, and wherein the sorting comprises sorting via activation of the one or more microfluidic valves. In other embodiments, the microfluidic device comprises a selectively activatable droplet maker which forms droplets from a fluid stream, wherein the encapsulating is accomplished by the droplet maker. In some embodiments, the microfluidic device is integrated with an automated system which selectively positions the delivery orifice relative to the substrate, and wherein the method comprises selectively positioning via the automated system the delivery orifice relative to the substrate to selectively deliver the plurality of the discrete entities to the substrate. In additional embodiments, the microfluidic device is integrated with an automated system which selectively positions the substrate relative to the delivery orifice, and wherein the method comprises selectively positioning via the automated system the substrate relative to the delivery orifice to selectively deliver the plurality of discrete entities to the substrate. In particular embodiments, the substrate comprises individually controllable electrodes, wherein each of the electrodes, when activated with electricity to become an activated electrode, is capable of affixing a first discrete entity to a surface of the substrate when the first discrete entity is deposited in proximity so the activated electrode.

In some embodiments, the SD cells are eukaryotic cells (e.g., yeast cells). In certain embodiments, the TM cell(s) is/are eukaryotic cell(s) (e.g., mammalian cell). In particular embodiments, the TM cell(s) is/are human cell(s). In other embodiments, each of the plurality of affixed entities further comprises at least one of the following: a bead, primer, barcode sequence, template switching oligonucleotide (TSO), or a reverse transcriptase.

### DESCRIPTION OF THE FIGURES

FIG. 1A shows a schematic of Fab fragment composed of a light chain variable and constant regions linked to heavy chain variable and constant region 1 (CH1) as a single chain. The complementarity determining regions (CDRs) are synthetically varied to generate hundreds of thousands of unique Fab sequences with potential affinities for diverse targets, which are displayed on the surface display (SD) cells discussed herein. Other configurations of Abs such as Single-chain variable fragment (ScFv), bispecific antibodies can also be employed. FIG. 1B shows an exemplary embodiment for the cell-cell microfluidic embodiments disclosed herein that allows for screening antibody variable regions against transmembrane (TM) proteins. For example, as shown in this figure, GFP expressing yeast antibody libraries are encapsulated as single-cells, propagated, and merged with droplets containing single mammalian cells that express a TM protein target.

FIG. 2 shows an exemplary microfluidic system herein where single droplets containing a single cell (e.g., expressing at least one variable region on its surface) is flowed in a channel, sorted, and deposited on a substrate containing oil, such that the droplet is affixed to the substrate via dielectrophoresis due to the electrodes in the substrate.

FIG. 3 shows an exemplary microfluidic system herein where single droplets containing a single cell (e.g., over-expressing a transmembrane protein of interest) is flowed in a channel, sorted, and deposited on a substrate containing oil, such that the droplet merges with the droplet already present as discussed in FIG. 2.

FIG. 4A shows an exemplary system composed of a flow channel for droplets that has a detector and waste tube, which flows droplets to a print head so the droplets can be deposited on a multi-array substrate which is covered by oil. FIG. 4B shows a multi-array substrate with hundreds of electrodes for individually affixing droplets to the substrate. FIG. 4C shows a single exemplary electrode present in the substrate.

### DETAILED DESCRIPTION

The present invention provides systems, kits, and methods for analyzing cell-cell interactions, such as transmembrane proteins binding to surface displayed variable regions, via discrete entity (e.g., droplet) microfluidics. In certain embodiments, a plurality of first discrete entities and a plurality of second discrete entities are merged on a substrate to generate a plurality of merged fixed entities (e.g., fixed via an electrical force), each of which contains one cell expressing a transmembrane (TM) protein and labeled clonal cells displaying a heterologous antibody variable region. In certain embodiments, any binding of the clonal cells to the TM expressing cell is detected in each merged fixed entity, and the clonal cells found to bind are treated in order to sequence the nucleic acid encoding the variable region.

In certain embodiments, Printed Droplet Microfluidics (PDM) technology is employed (e.g., as described in US Pat. Pub. 2018/0056288; Cole et al., PNAS, 2017, 114(33):8728-8733; and Siltanen et al., Scientific Reports, 2018, 8:7913, all of which are herein incorporated by reference in their entireties) to encapsulate detectable-labeled single eukaryotic cells (e.g., yeast cells) that express a unique antibody variable region species, propagate them into isogenic colonies, and deterministically merge with droplets containing a single target cell (e.g., over-expressing a transmembrane protein of interest). If the antibody variable region is for the target, the small eukaryotic cells will, for example, surface decorate the single target cell (e.g., human cell) similar to the head/ray floret arrangement of a sunflower. Eukaryotic cells that form this configuration are identifiable, for example, via imaging or flow cytometry and will be either selectively sequenced or the antibody sequence identified post-sequencing using positional barcodes or similar methods. In certain embodiments, the droplet microfluidic platform can encapsulate single yeast cells from a library of yeast expressing hundreds of thousands of antibody variable region variants and a detectable protein (e.g., green fluorescent protein). The antibody variable variants can, for example, be Fab fragments or nanobodies. These single yeast cells are propagated in individual droplets and then merged with single mammalian cells that express the TM protein of interest. Binders are imaged and sequenced to identify the antibody sequences that are hits.

In some embodiments, the detectable protein (DP)-expressing yeast cells are modified to express Fab fragments where each yeast cell would express a unique Fab sequence (McMahon et al., Nat. Struct. Mol. Biol., vol. 25, no. 3, pp. 289-296, 2018, herein incorporated by reference). Single-DP-Fab-yeast cells are encapsulated in droplets and propagated (e.g., for 2-3 cell divisions). Using PDM, human cells expressing a specific TM target putatively in its native conformation is merged with each clonal droplet of yeast cells, incubated and imaged. In certain embodiments, the yeast cells expressing binders bind the larger human cell and the fluorescence quantitation resulting in a sharper peak as opposed to the non-binders, which show a more diffuse signal indicative of the yeast cells dispersed throughout the droplet. Mammalian cells expressing a reporter can also be used that specifically interrogates GPCR-target interaction in coculture and the specific interactors can be sorted based on reporter signal intensity for sequencing. Instead of yeast cells expressing antibody libraries, other systems such as, for example, phage display libraries or baculovirus transfected Spodoptera frugiperda libraries can also be used.

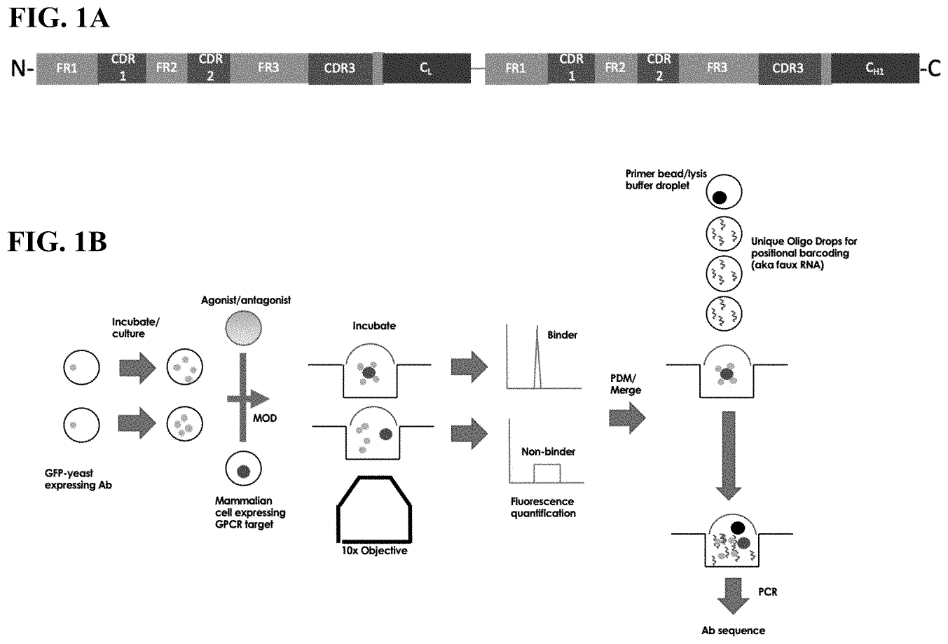

The following provides an exemplary protocol as generally shown in FIG. 1B. First, a mammalian cell-line is obtained that over-expresses a transmembrane (TM) protein of interest, such as a G-protein coupled receptor (GPCR). Hundreds of such cell-lines are commercially available from Creative Biogene (Shirley, N.Y.). A partial list of such cell lines is shown in Table 1 below.

TABLE 1

| | |
|---|---|
| CSC-RG0001 | Human CNR2 Stable Cell Line-CHO |
| CSC-RG0002 | Human BDKRB1 Stable Cell Line-HEK293 |
| CSC-RG0003 | Human CNR1 Stable Cell Line-HEK293 |
| CSC-RG0004 | Human ADRB3 Stable Cell Line-HEK293 |
| CSC-RG0005 | Human CHRM1 Stable Cell Line-U2OS |
| CSC-RG0006 | Human BDKRB2 Stable Cell Line-U2OS |
| CSC-RG0007 | Human BDKRB2 Stable Cell Line-HEK293 |
| CSC-RG0008 | Human CALCR Stable Cell Line-HEK293 |
| CSC-RG0009 | Human CRHR2 Stable Cell Line-HEK293 |
| CSC-RG0010 | Human FSHR Stable Cell Line-HEK293 |
| CSC-RG0011 | Human ADCYAP1R1 Stable Cell Line-HEK293 |
| CSC-RG0012 | Human ADRA1A Stable Cell Line-U2OS |
| CSC-RG0013 | Human TACR2 Stable Cell Line-U2OS |
| CSC-RG0014 | Human CHRM3 Stable Cell Line-U2OS |
| CSC-RG0015 | Human CHRM5 Stable Cell Line-U2OS |
| CSC-RG0016 | Human TACR3 Stable Cell Line-U2OS |
| CSC-RG0017 | Human TACR1 Stable Cell Line-HEK293 |
| CSC-RG0018 | Human AVPR1A Stable Cell Line-HEK293 |
| CSC-RG0019 | Human ADRA1B Stable Cell Line-U2OS |
| CSC-RG0020 | Human GRPR Stable Cell Line-U2OS |

Next, a population of yeast cells with membrane bound variable region or antibodies (e.g., from a library) are constructed. Yeast cells expressing antibody chains fused to a detectable protein, such as GFP, are constructed (e.g., employing the methods in D. Huang and E. V. Shusta, “A yeast platform for the production of single-chain antibody-green fluorescent protein fusions,” Appl. Environ. Microbiol., vol. 72, no. 12, pp. 7748-7759, 2006, herein incorporated by reference). Next, yeast cells are encapsulated in droplets using printed droplet microfluidics (PDM). Yeast cells have been successfully encapsulated in droplets and cultured using the printed droplet microfluidics platform (Siltanen et al., Scientific Reports, 2018, 8:7913, herein incorporated by reference). PDM has also been used to merge cell containing droplets to perform cocultures in nanowell arrays and image them (see, e.g., US Pat. Pub. 2018/0056288; Cole et al., PNAS, 2017, 114(33):8728-8733; and Siltanen et al., Scientific Reports, 2018, 8:7913; all of which are herein incorporated by reference in their entireties). PDM is a flexible technology that can be used to encapsulate not only cells but also fluorescent reagents and beads. Thus, peptides and other agonists/antagonists can be encapsulated and selectively merged to form cocultures with yeast-GFP antibody libraries, mammalian cells expressing GPCRs or other transmembrane proteins. Next, the substrate is prepared, such as in 2018/0056288; Cole et al., and Siltanen et al. Next, imaging and signal-based sorting is conducted. The assembled cocultures are, for example, imaged using a 4× or 10× objective and analyzed using ImageJ software configured to PDM. Nanowells displaying positive signals are recorded. Droplets containing lysis buffer/Dropseq beads [see, e.g., Macosko et al., “Highly Parallel Genome-wide Expression Profiling of Individual Cells Using Nanoliter Droplets,” Cell, vol. 161, no. 5, pp. 1202-1214, 2015, herein incorporated by reference) and unique oligo drops for positional barcoding are sorted and merged into the droplets in the nanowells. The mammalian and yeast cells are lysed, and the mRNA/positional barcodes are captured on the Dropseq beads. The droplets are broken, Dropseq beads retrieved, washed and subjected to reverse transcription. The reverse transcribed products are further amplified using antibody specific primers according to previously published methods (e.g., McMahon et al., “Yeast surface display platform for rapid discovery of conformationally selective nanobodies,” Nat. Struct. Mol. Biol., vol. 25, no. 3, pp. 289-296, 2018, herein incorporated by reference) and sequenced to determine the specific antibody sequence that interacted with the GPCR.

In certain embodiments, overlap extension PCR is employed to both amplify nucleic acid from SD cells and TM cells, but also to associate the nucleic acid encoding the variable region from the SD cell with the nucleic acid encoding the transmembrane protein (e.g., GPCR) from the TM cell. For example, after imaging a droplet and finding SD cells bound to a TM cell, such cells could be lysed releasing the nucleic acid. To such droplets, Drop-seq beads and RT-PCR reagents are added to capture the mRNAs on the Drop-seq bead. The PCR could use overlap extension PCR to associate the variable region nucleic acid with the transmembrane protein nucleic acid. Details on overlap extension PCR are found in the art, including at, for example, U.S. Pat. Pub. 20150154352 and Turchaninova et al., Eur. J. Immunol. 2013. 43: 2507-2515, both of which are herein incorporated by reference. One of the advantages of this method is there will not be a need for barcoding individual assay positive droplets because the variable region (e.g., two Fab fragment) and the transmembrane protein (e.g., GPCR) identify will be linked in one fragment.

In general, provided herein are methods for flowing discrete entities (e.g., droplets) through a microfluidic device in a carrier fluid, such as a carrier fluid in which the discrete entities are insoluble and/or immiscible, and directing the carrier fluid and one or more of the discrete entities through a portion of a microfluidic device, such as a delivery orifice, to a substrate and/or affixing the one or more discrete entities to a substrate. Discrete entities may be affixed to a substrate, for example, by one or more forces, such as an electrical (e.g., dielectrophoretic), gravitational, and/or magnetic force. Discrete entities as used or generated in connection with the subject methods, devices, and/or systems may be, for example, sphere shaped or they may have any other suitable shape, e.g., an ovular or oblong shape. Discrete entities as described herein may include a liquid phase and/or a solid phase material. In some embodiments, discrete entities according to the present disclosure include a gel material. In some embodiments, the subject discrete entities have a dimension, e.g., a diameter, of or about 1.0 μm to 1000 μm, inclusive, such as 1.0 μm to 750 μm, 1.0 μm to 500 μm, 1.0 μm to 100 μm, 1.0 μm to 10 μm, or 1.0 μm to 5 μm, inclusive. In some embodiments, discrete entities as described herein have a dimension, e.g., diameter, of or about 1.0 μm to 5 μm, 5 pm to 10 μm, 10 μm to 100 μm, 100 μm to 500 μm, 500 μm to 750 μm, or 750 μm to 1000 μm, inclusive. Furthermore, in some embodiments, discrete entities as described herein have a volume ranging from about 1 fL to 1 nL, inclusive, such as from 1 fL to 100 pL, 1 fL to 10 pL, 1 fL to 1 pL, 1 fL to 100 fL, or 1 fL to 10 fL, inclusive. In some embodiments, discrete entities as described herein have a volume of 1 fL to 10 fL, 10 fL to 100 fL, 100 fL to 1 pL, 1 pL to 10 pL, 10 pL to 100 pL or 100 pL to 1 nL, inclusive. In addition, discrete entities as described herein may have a size and/or shape such that they may be produced in, on, or by a microfluidic device and/or flowed from or applied by a microfluidic device.

In some embodiments, the discrete entities as described herein are droplets. The terms “drop,” “droplet,” and “microdroplet” are used interchangeably herein, to refer to small, generally spherically structures, containing at least a first fluid phase, e.g., an aqueous phase (e.g., water), bounded by a second fluid phase (e.g., oil) which is immiscible with the first fluid phase. In some embodiments, droplets herein may contain a first fluid phase, e,g, oil, bounded by a second immiscible fluid phase, e.g. an aqueous phase fluid (e.g, water). In some embodiments, the second fluid phase will be an immiscible phase carrier fluid. Thus, droplets herein may be provided as aqueous-in-oil emulsions or oil-in-aqueous emulsions. Droplets may be sized and/or shaped as described herein for discrete entities. For example, droplets according to the present disclosure generally range from 1 μm to 1000 μm, inclusive, in diameter. Droplets according to the present disclosure may be used to encapsulate cells, nucleic acids (e.g., DNA), enzymes, reagents, and a variety of other components. The term droplet may be used to refer to a droplet produced in, on, or by a microfluidic device and/or flowed from or applied by a microfluidic device.

As used herein, the term “carrier fluid” refers to a fluid configured or selected to contain one or more discrete entities, e.g., droplets, as described herein. A carrier fluid may include one or more substances and may have one or more properties, e.g., viscosity, which allow it to be flowed through a microfluidic device or a portion thereof, such as a delivery orifice. In some embodiments, carrier fluids include, for example: oil or water, and may be in a liquid or gas phase.

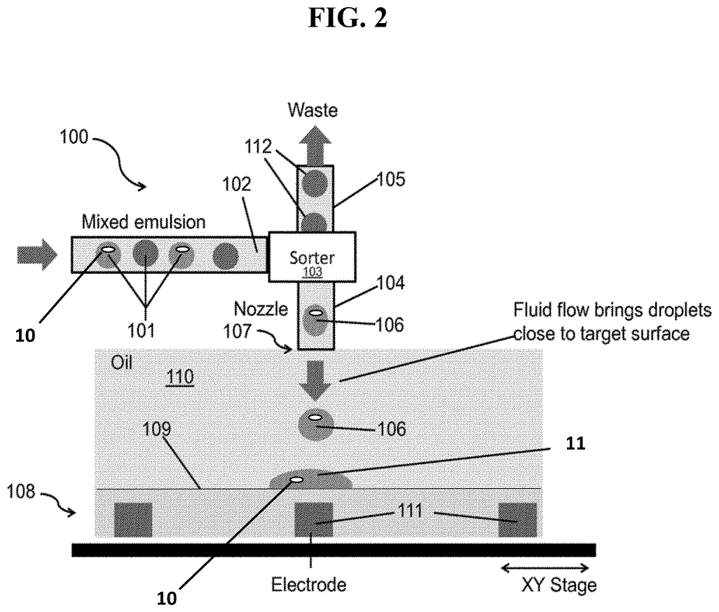

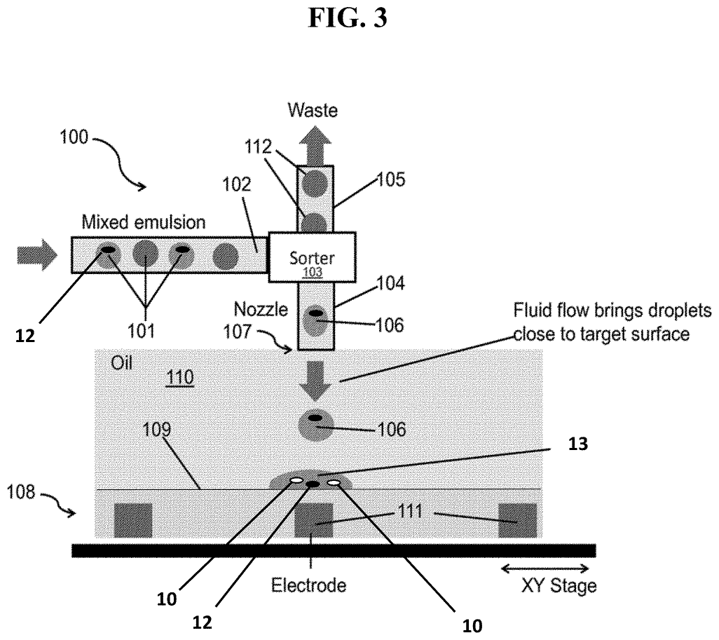

FIGS. 2 and 3 presents a non-limiting, simplified representation of one type of a microfluidics system and method according to the present disclosure. The particular embodiment depicted in FIG. 2 shows the delivery of discrete entities (droplets are illustrated by way of example) to a substrate, where the discrete entity 106 contains a single surface display (SD) cell 10 displaying a surface protein comprising an antibody variable region. FIG. 3 shows a similar embodiments, except with a discrete entity 106 with a transmembrane protein cell (TM cell) 12 that over-expresses a transmembrane protein. In one such method, discrete entities 101, e.g., droplets, are prepared using a device, e.g., a microfluidic device 100, and a carrier fluid 102 to produce a mixed emulsion including the discrete entities. A variety of suitable droplet makers are known in the art, which may be used to prepare the mixed emulsion, e.g., droplet makers described in PCT Publication No. WO 2014/028378, the disclosure of which is incorporated by reference herein in its entirety and for all purposes.

In some embodiments of the disclosed methods, microfluidic devices are utilized which include one or more droplet makers configured to form droplets from a fluid stream. Suitable droplet makers include selectively activatable droplet makers and the methods may include forming one or more discrete entities via selective activation of the droplet maker. The methods may also include forming discrete entities using a droplet maker, wherein the discrete entities include one or more entities which differ in composition.

Once prepared, a mixed emulsion, containing surface-display (SD) cell 10 or TM cell 12, may be moved, e.g., moved and/or flowed to another portion of the microfluidic device 100, such as a sorter 103. A subset of the discrete entities 101 may be separated using a sorter 103. A sorter 103 may be configured to detect and/or separate discrete entities, e.g., discrete entities present in a carrier fluid, having different types, e.g., different compositions and/or sizes, such as a first type, e.g., a type containing one or more cells of interest, and a second type, e.g., a type not containing one or more cells of interest. As such, a sorter 103 may provide one or more sorted discrete entities 106 (e.g., one or more discrete entities including an SC cell or TM cell) and direct them via a first channel 104 to a nozzle including a delivery orifice 107 for delivery to a substrate 108. A sorter 103 may also provide one or more sorted discrete entities 112 (e.g., one or more discrete entities not including a cell) and direct them via a second channel 105 to a waste outlet.

In some embodiments, the discrete entities not sorted for delivery via a delivery orifice, are recovered and/or recycled by, for example, being re-injected into the carrier fluid upstream of the sorter 103. Various embodiments of the methods disclosed herein include repeated recycling of discrete entities not selected for delivery through the delivery orifice in a particular pass through the sorter. Sorting, according to the subject embodiments, is described in further detail below. Also, in various embodiments, one or more discrete entities, e.g., all the discrete entities present in a mixed emulsion, remain contained e.g., encapsulated, in a carrier fluid, e.g., a hydrophobic solution (e.g., oil), or a hydrophilic solution (e.g., an aqueous solution), prior to sorting and/or throughout a sorting process carried out by the sorter 103 and/or throughout the process of directing the one or more entities through a portion of a microfluidic device, e.g., a delivery orifice, and/or throughout a process of affixing the entities to a substrate.

As discussed above, a sorted subset of discrete entities of interest, e.g., discrete entities 106, (e.g., discrete entities containing a cells of interest), may in some embodiments, be directed through a delivery orifice 107 of a microfluidic device 100 to a substrate 108. In some embodiments of the methods, a microfluidic device 100, or a portion thereof, e.g., a delivery orifice 107, contacts a substrate, e.g., a substrate 108, or a portion thereof, to which it delivers discrete entities. In other embodiments, a microfluidic device 100, or a portion thereof, e.g., a delivery orifice 107, delivers discrete entities to a substrate, e.g., a substrate 108, or a portion thereof, by dispensing the discrete entities in a carrier fluid, e.g., a carrier fluid 102, in proximity to a surface of the substrate, for example into a fluid on the surface of the substrate (e.g., substrate fluid 110), which fluid is miscible with the carrier fluid and immiscible with the discrete entities.

A delivery orifice as described herein, e.g., a delivery orifice of a microfluidic nozzle as described herein, will generally have dimensions that are similar to the size of the droplets to be delivered therethrough. Accordingly, in some embodiments, a delivery orifice as described herein has a diameter of from about 1 μm to about 1000 μm, inclusive, e.g., from about 10 μm to about 300 μm, inclusive. In some embodiments, a delivery orifice as described herein has a diameter of from about 1 μm to about 10 μm, from about 10 μm to about 100 μm, from about 100 μm to about 500 μm, or from about 500 μm to about 1000 μm, inclusive. The nozzle can be molded as part of a microfluidic sorter as described herein, or can be a separate part that is mated with a microfluidic sorter as described herein. Suitable materials for the nozzle may include, e.g., polymeric tubing, small bore hypodermic tubing, and modified glass capillaries.

In some embodiments of the subject systems, devices and methods, a microfluidic device including a sorting junction is employed to apply and sort a mixed emulsion in order to deliver select discrete entities, e.g., droplets, to a delivery orifice, e.g., a delivery orifice of a print head. The microfluidic device utilizes a moat salt solution (to generate the field gradient used for dielectrophoretic deflection and to limit stray fields that can cause unintended droplet merger), spacer oil, and an electrode salt solution to facilitate sorting. The microfluidic device depicted provides junctions including a reinjection junction for providing a discrete entity-containing emulsion to be sorted and a sorting junction including a sorter for sorting, e.g., by making positive and negative sorts of discrete entities. Sorting may be accomplished, e.g., by applying an electric field via an electrode, e.g., a liquid electrode including, e.g., an electrode salt solution.

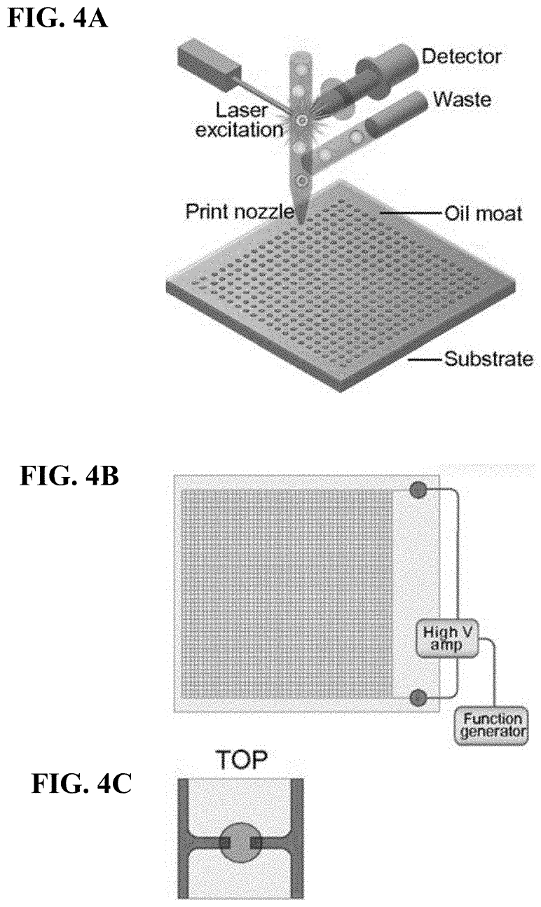

In certain embodiments, liquid electrodes include liquid electrode channels filled with a conducting liquid (e.g. salt water or buffer) and situated at positions in the microfluidic device where an electric field is desired. In particular embodiments, the liquid electrodes (e.g., as pictured in FIGS. 4B and 4C) are energized using a power supply or high voltage amplifier. In some embodiments, the liquid electrode channel includes an inlet port so that a conducting liquid can be added to the liquid electrode channel. Such conducting liquid may be added to the liquid electrode channel, for example, by connecting a tube filled with the liquid to the inlet port and applying pressure. In particular embodiments, the liquid electrode channel also includes an outlet port for releasing conducting liquid from the channel. The microfluidic device, as shown in FIG. 4A, also includes outlets to one or more print nozzles and to a waste container or channel.

As discussed above, some embodiments, such as those described in connection with FIGS. 2 and 3, include affixing one or more discrete entities 101 to a substrate 108. Substrate 108 includes a surface, e.g., a surface 109, upon which a layer of fluid, e.g., substrate fluid 110, e.g., oil, may be provided or deposited. Suitable substrate fluids may include, for example, one or more liquids in which discrete entities are insoluble and/or immiscible, such as water and/or oil depending on the nature of the discrete entities. Substrate fluids may be the same type of fluid as a carrier fluid, e.g., a fluid having the same composition as a carrier fluid, e.g., a fluid including water and/or oil, or may be a different type of fluid than the carrier fluid, e.g., a fluid including water and/or oil.

In some embodiments, the disclosed methods may include moving one or more discrete entities through a device and/or affixing one or more discrete entities to a substrate and/or removing the discrete entities from the substrate by changing the buoyancy of the discrete entities and/or exerting one or more forces on one or more components, e.g., beads, of the discrete entities. Embodiments of the methods also include releasing one or more discrete entities, e.g., an affixed discrete entity, from a substrate by, for example, modulating, e.g., modulating by removing, one or more force affixing the entity to the substrate. In some instances, discrete entities are removed from a substrate by removing an electric field affixing them thereto.

To facilitate the above manipulations, the present disclosure provides, in some embodiments, a substrate which includes an array of individually controllable electrodes. Such substrates may be configured such that individual electrodes in the array can be selectively activated and deactivated, e.g., by applying or removing a voltage or current to the selected electrode. In this manner, a specific discrete entity affixed via a force applied by the electrode may be selectively released from a substrate surface, while unselected discrete entities remain affixed via application of the force. The electrodes of such an array may be embedded in a substrate material (e.g., a suitable polymer material), e.g., beneath a surface of the substrate to which the discrete entities are affixed via application of the force. A variety of suitable conductive materials are known in the art which may be utilized in connection with the disclosed electrode arrays, including various metals. Liquid electrodes as described previously herein may also be used for such an application.

The methods also include methods of adding reagents to a discrete entity, e.g., a droplet, e.g., a droplet affixed to a substrate. Such methods may include delivering and/or affixing a first discrete entity (e.g., having an SD cell) in a first carrier fluid to a substrate or a portion thereof, e.g., a substrate surface. The methods may also include delivering one or more other discrete entities (e.g., having a TM cell), e.g., a second droplet, such as a discrete entity in a second carrier fluid and/or including one or more reagents, to a location on the substrate which is the same location as the first discrete entity or a location adjacent or in proximity to that of the first discrete entity. The first and subsequent applied discrete entities may then be coalesced such that the contents, including, for example, one or more reagents, of the first and subsequent discrete entities are combined. In some embodiments, coalescence is spontaneous and in other embodiments, coalescing discrete entities includes applying a force, such as an electrical force, to one or more of the discrete entities. For example, applying an electrical field to two or more droplets in close proximity can induce dynamic instability at the oil-water interfaces that results in droplet merger to reduce the surface energy of the oil-water system. The first and second carrier fluids, as described above, may be the same type of fluid or different types of fluids.

Some embodiments of this disclosure also include methods of adding one or more reagent and/or components, such as one or more beads, to one or more discrete entities, e.g., droplets, by delivering one or more discrete entities in a first carrier fluid via a first orifice of a device to a surface of a substrate. The methods may also include positioning one or more of such discrete entities in a second carrier fluid, e.g., a carrier fluid which is of the same or a different type than the first carrier fluid, on the substrate surface and/or affixing the discrete entities to the substrate via a force. According to the subject methods, an orifice of a device, such as an orifice operably connected, e.g., fluidically connected, to a reagent source, may then be inserted into one or more of the affixed discrete entities. Upon insertion, the orifice may be utilized to inject one or more reagents into the one or more discrete entities.

Embodiments of the methods may include modulating the environment of a discrete entity and thereby modulating the contents of the discrete entity, e.g., by adding and/or removing contents of the droplet. Such modulation may include modulating a temperature, pH, pressure, chemical composition, and/or radiation level of an environment of one or more discrete entities. Such modulation may also be of the immediate environment of one or more discrete entities, such as an emulsion in which the discrete entities are provided and/or one or more space, such as a conduit, channel, or container, within a microfluidic device. An immediate environment of a discrete entity which may be modulated may also include a fluid volume, such as a fluid flow, in which the discrete entity is provided. One or more discrete entities may also be stored in a modulated environment.

The methods of this disclosure may also include recovering all or a portion of one or more discrete entities which have been affixed to a substrate (e.g., recovering a merged affixed entity). For example, one or more materials, such as one or more solvents and/or reagents may be recovered from a droplet via, for example, extraction. Such a recovery may be conducted by contacting one or more affixed discrete entities with a portion of a device, such as a microfluidic orifice connected to a suction device for sucking one or more material, such as one or more solvent and/or reagent from one or more affixed discrete entity. A microfluidic orifice may be inserted into a discrete entity and/or placed in proximity to a discrete entity, e.g., placed at a distance from a discrete entity having an order of magnitude of a discrete entity or smaller, for performing recovery from the entity. Embodiments of the methods of recovery from a discrete entity may also include shearing, e.g., detaching, a discrete entity from a substrate surface by, e.g., increasing the buoyancy of one or more discrete entities. The buoyancy of a discrete entity can be increased by increasing the volume of the discrete entity by, for example, injecting aqueous fluid or non-aqueous fluid into the discrete entity.

In some embodiments, the methods may include concentrating one or more components (e.g., beads) present in a discrete entity at a location within a discrete entity. Such concentrated components or alternatively, portions of the discrete entity not containing the components, may then be selectively removed (e.g., removed by suction, from the discrete entity). One or more components removed from discrete entities may then be conveyed into one or more isolated containers via, for example, a delivery orifice.

In various aspects, substrates for use in connection with the disclosed methods include one or more channels filled with one or more conductive, e.g., electrically conductive, liquid or solid materials, e.g., an electrode material. In some embodiments, such substrates may also include an insulating sheet positioned between the channels and the carrier fluid. In some embodiments, one or more channels are configured, e.g., patterned, to generate an electric field above a portion of a substrate, such as an insulating sheet, upon application of a voltage to the one or more channels. In some embodiments, such a voltage and a resulting electrical field or an aspect thereof, e.g., a dielectrophoretic force, is sufficient to affix one or more discrete entities to the substrate. In some embodiments, a substrate, or a portion thereof, includes one or more electrodes having a net charge which is opposite in polarity, e.g., negative or positive, relative to the polarity of one or more discrete entities, e.g., droplets, being affixed to the substrate.

In some embodiments, surfaces of substrates include one or more electrodes. In various embodiments, one or more electrodes are pre-formed on a substrate or portion thereof, e.g., a substrate surface. Substrates may, in various embodiments, be mounted upon and/or adjacently to, e.g., contacting, a stage, such as a movable stage, such as a stage movable in an X-Y and/or Z direction. In some embodiments, a stage is movable in a direction toward and/or or away from a microfluidic device, or a portion thereof, e.g., a delivery orifice. Also, in some embodiments, a microfluidic device, or a portion thereof, e.g., a delivery orifice is movable in a direction toward and/or or away from another portion of a device, e.g., a stage, and/or a substrate. A stage and/or a microfluidic device, or a portion thereof, e.g., a delivery orifice, may be movable in constant movement or in increments on a scale of a diameter or radius of one or more discrete entities, e.g., 5 or less, 10 or less, 50 or less, or 100 or less discrete entities. A stage and/or a microfluidic device, or a portion thereof, may be movable in one or more direction, e.g., an X and/or Y and/or Z direction, in one or more increments having a distance of, for example, 1 μm to 1000 μm, inclusive, such as 1.0 μm to 750 μm, 10 μm to 500 μm, 1 μm to 50 μm, or 1 μm to 10 μm, inclusive. In some embodiments, the devices may me movable in constant movement or one or more increments on a scale to correspond with positions on a substrate where discrete entities may be attached, such as wells on a well plate including any of the well plates described herein.

In some embodiments, the methods include affixing one or more discrete entities 101 to a substrate 108, or a portion thereof, e.g., a surface 109, via wetting, e.g., electrowetting. In some embodiments, wetting includes moving, e.g., flowing, one or more discrete entities 106 from a delivery orifice 107, through a substrate fluid 110, to a substrate surface 109 of a substrate. In some embodiments, the wettability of a substrate is sufficient to attach one or more discrete entities to the substrate via, for example, wetting forces. In some embodiments, the methods include modifying, e.g., increasing or decreasing, the wettability of a substrate so as to be sufficient to affix a discrete entity to the substrate via wetting forces. Various aspects of the methods may also include applying exogenous electromagnetic radiation in an amount sufficient to affix a discrete entity to a specific location on a substrate.

In some embodiments, the subject methods include patterning one or more channels, e.g., channels of a substrate or aspects thereof, to provide a plurality of charged electrode features in a grid pattern. Such an arrangement is shown, for example, in FIG. 4B, which depicts a substrate, including electrode features (see FIG. 4C). A nozzle, shown in FIG. 4A, including delivery orifice is shown. Droplets are affixed to the grid pattern using dielectrophoresis, which allows the application of forces to uncharged conductive droplets suspended in a nonconductive medium. For example, in one embodiment, unaffixed droplets experience a net force towards the regions on the surface of the substrate with the highest electric field gradient, the gap between oppositely charged features (FIG. 4C). Once droplets are brought to the substrate surface, the surfactant layer stabilizing the droplets is disrupted, and the droplet wets the region. Due to the abrupt changes in geometry, the highest electric field gradients occur at the boundaries between charged features and the gap. Droplets wetting the region experience a lateral force towards the charged features, which causes a flattening and elongation of the droplet that is proportionate to the applied electric field. The charged features, do not necessarily need to have constant and opposite polarities. For example, high electric field gradients can be created by electrifying the charged feature with a high voltage AC signal (1.5 kV, 30 kHZ) while grounding the other feature.

As illustrated in FIGS. 2 and 3, affixing one or more discrete entities, e.g., discrete entities 101, to a substrate, e.g., a substrate 108, or a portion thereof, e.g., a surface 109, may include attaching the discrete entities to the substrate, e.g., substrate 108, via a force, such as a gravitational, electrical, and/or magnetic force. As such, in some embodiments, a delivery orifice, e.g., a delivery orifice 107, is positioned above a substrate 108. In some embodiments, the methods include applying an electrical voltage and/or current to electrodes, e.g., electrodes 111, positioned in or on the substrate, e.g., substrate 108. Affixing one or more discrete entities, e.g., discrete entities 101, to a substrate, e.g., a substrate 108, or a portion thereof, may also include affixing the entities to the substrate via interfacial tension.

In some embodiments of the disclosed methods, one or more microfluidic devices are integrated with an automated system which selectively positions one or more portions of the microfluidic devices, e.g., one or more delivery orifices, relative to a substrate or a portion thereof, e.g., a substrate surface. Accordingly, in some embodiments the methods include selectively positioning, e.g., positioning at a particular location using an automated system, one or more delivery orifices relative to a substrate or a portion of a substrate to selectively deliver one or more discrete entities to one or more locations on or in proximity to the substrate or a portion thereof, e.g., a substrate surface. Automated systems as disclosed may include one or more control units, e.g., control units including a central processing unit, to control one or more aspects of applying discrete entities to a substrate, such as physical positioning of one or more delivery orifice and/or timing of discrete entity dispensing. Automated systems may be configured to position, e.g., position independently, one or more delivery orifices with respect to a stationary substrate or position a substrate with respect to one or more stationary delivery orifices. Aspects of the subject methods may include delivering a first member of a plurality of discrete entities to a first location on or in proximity to a substrate or a portion thereof, e.g., a substrate surface, and a second member of the plurality of discrete entities to the first location or a second location on or in proximity to the substrate.

The methods may also include modulating, e.g., changing one or more aspect of, one or more force, e.g., by modulating an electric field and/or buoyancy of a discrete entity in one or more carrier solution, to thereby move one or more discrete entities, e.g., a droplet, from a first affixed location on a substrate to another location. The methods may also include applying one or more additional, e.g., second, force which is sufficient to move one or more discrete entities from a first affixed location to a second location on a substrate and/or affix the one or more discrete entities at the second location. Aspects of the methods may also include applying a cross flow of fluid and/or exogenous electromagnetic radiation sufficient to move a discrete entity from a first location, e.g., a first affixed location, on a substrate to a second location on a substrate.

Embodiments of the methods may also include performing one or more assays, e.g., one or more biological assays, such as any of the assays described herein, on and/or in one or more of the discrete entities before and/or after delivery of a discrete entity to a substrate or a portion thereof, e.g., a substrate surface. In some embodiments, such substrates may include a well plate or a portion thereof The term “well plate”, is used broadly herein, to refer to a plate having one or more wells, e.g., divots or compartments, therein, such as a mictrotiter plate. However, as used herein, the term “well plate” may also refer to a patterned array of discrete entities, e.g., droplets, as described herein, which discrete entities are affixed to a substrate surface. In such embodiments, the substrate surface may include traditional wells, such as divots or compartments, but may alternatively be a flat surface. Well plates which may be prepared and/or utilized in accordance with the subject methods and devices, e.g., well plates including ordered arrays of discrete entities, may include well plates having, for example, from 20,000 to 500,000, inclusive, wells, such as from 50,000 to 150,000, inclusive, such as from 80,000 to 120,000, inclusive, such as 100,000 wells. In certain well plates, each well may have an area ranging, for example, from 0.01 mm2 to 1 mm2, inclusive, such as from 0.05 mm2 to 0.5 mm2, such as about 0.10 mm2.

Aspects of the disclosed methods may also include controlling, e.g., maintaining, the temperature of one or more discrete entities before and/or after delivery of the one or more entities to a substrate or a portion thereof, e.g., a substrate surface. For example, in some embodiments, one or more discrete entities are thermalcycled before and/or after delivery to a substrate or a portion thereof, e.g., a substrate surface.

In certain embodiments of the present disclosure, an emulsion including droplets of different composition is “printed” to a substrate using a microfluidic print head, e.g., as described herein. The droplets are made ahead of time using a microfluidic or non-microfluidic technique, such as flow focusing or membrane emulsification, respectively. The pre-formed droplets are then introduced into the print head and sorted on demand according to their fluorescence. In certain embodiments, the droplet solutions are dyed with different solutions, or have a cell with detectable protein, prior to being encapsulated as droplets so that, when injected into the print head, a detection technique, such as flow dropometry, can be used to identify each droplet's type and, using this information, a computer can determine which droplets to sort. This allows dispensing of precise solutions to the substrate. Once dispensed by the print head, the droplets are affixed to the substrate using a force such as, for example, a dielectrophoretic force that is generated via electrodes fabricated under the substrate surface. A layer of oil above the substrate allows the droplets to remain in the carrier fluid at all times, that is, the droplets are in the carrier fluid after generation, flowed via carrier fluid throughout the print head, and then dispensed into a carrier-fluid coated substrate. This keeps the droplets encapsulated at all times and protects against evaporation.

In addition to dielectrophoresis, other forces can also be applied to affix the droplets. For example, an electrical force can be applied in which the substrate can be charged oppositely to the droplets, creating an electrical attraction. The droplets can be charged as they pass through the microfluidic print head using a channel comprising charged fluid that contacts the droplets or, for example, a salt water electrode as described herein. Other forces that can be used are, for example, gravitational, in which the density of the droplets being larger than that of the carrier fluid causes them to sink into a well patterned on the substrate or float into a an upside-down well, if less dense than the carrier fluid. Magnetic forces can be used in similar ways. Wetting and chemical forces can also be used such that the droplets, upon contacting the substrate, wet the surface and are adhered to it via surface tension.

In microfluidic and other applications it is often desirable to generate droplets of defined type on demand (e.g., droplets containing an SD cell and a droplet containing a TM cells). One method for accomplishing this is using a microfluidic droplet generator controlled by a membrane valve. When the valve is closed, the dispersed phase does not flow and no droplets are generated. When it is opened, it flows and droplets are generated. This approach can generate droplets on demand as fast as the valve can opened and closed (e.g., 100 Hz). In addition, the droplets are all formed of the same fluid. To allow for generation of droplets on demand from multiple fluids, multiple devices, each with its own fluid, may be interfaced together. In certain embodiments of the present disclosure, the droplets are generated on demand by sorting them, from a preexisting emulsion, on demand. The droplets of the different desired fluids are first emulsified separately and combined into a single mixed emulsion. They are labeled to enable them to be differentiated from one another using optical detection, such as flow cytometry. This combined emulsion is then injected into a microfluidic sorter which scans the droplets and sorts them down two channels, a dispensing channel and a waste channel. From the perspective of the dispensing channel, this system includes a droplet on demand technique since, by diverting droplets down the dispensing channel on demand, droplets are ejected from the channel on demand. The emulsion that is sent into waste can be recycled through the sorter, to conserve reagents. The value of this droplet on demand technique is that it is limited in speed to the rate at which the droplets can be sorted. With sorting geometries incorporating gapped dividers between the sorting outlets, it is possible to sort droplets at >30 kHz, which is more than two orders of magnitude faster than can be achieved with other droplet on demand techniques.

There are a variety of substrates that can be constructed for trapping the droplets. One such substrate uses dielectrophoresis to trap the droplets. To generate the dielectrophoretic traps, the substrate may be fabricated so as to contain electrodes with which to generate the requisite electric fields. This can be accomplished by patterning electrodes under a dielectric sheet. The electrodes can be energized with positive and negative charges to generate large electric fields with a spatial gradient. When droplets are dispensed above the substrate and in the region of the field, dielectrophoretic forces will cause them to be attracted to the substrate, and adhere. The electrodes can be patterned using conventional fabrication techniques, such as metal sputtering or deposition on the sheet, or by fabricating microfluidic channels that can be bonded face-side-up to the bottom side of the sheet such that the channels are below the sheet and not in fluidic communication with the fluids above the sheet. The channels can then be filled with conductive medium, such as solder or electrolyte solution and charged to generate the desired electric fields for dielectrophoretic droplet tapping. By modulating the shapes, widths, and heights of the microfluidic channels, it is possible to structure the electrodes, thereby providing control over the fields that are applied to the droplets above the sheet.

In some embodiments, it is desirable to affix liquid or solid entities to the surface of a substrate via application of a force. One such force that can be used is dielectrophoresis, in which a patterned array of electrodes under the dielectric substrate is used to generate electric fields that dielectrophoretically attract or repel the entities, trapping them at the desired locations. Non-dielectrophoretic electrical forces can also be used. In such embodiments, the entities can be charged with, for example, a positive charge, either before, during, or after their flow through the print head. The substrate can then be charged oppositely, creating an electrical attraction between the entities and the substrate that will affix them. The polarity of the entities and substrate can also be modulated to generate a repulsive force allowing, for example, droplets to be ejected from the substrate. Electrodes can be used for these purposes. For example, the substrate can be uniformly charged with one polarity so that droplets of the opposite polarity will stick to the substrate. Provided a dielectric separates the electrode from the droplets, no charge will flow between the two and the force will remain. Alternatively, if the two are allowed to come into electrical contact, then charge will flow, removing the force but allowing, for example, a droplet to wet to the electrode and be affixed by interfacial tension forces.

In another embodiment, the electrodes can be patterned so that each trap has a single or multiple electrodes with the same or different polarity and charge. This can be used, for example, to generate dielectrophoretic traps appropriate to affix single droplets. Each electrode can be addressable and a large array of the traps can be fabricated into the substrate, allowing each drop to be switched on or off as desired. This can be used, for example, to capture droplets to specific traps by modulating the strength of the field of the trap where the droplet is to be affixed relative to other traps in the vicinity. The traps can also be turned off, to selectively release drops.

Different affixing forces are also possible, such as wettability and interfacial tension forces. In such embodiments, the substrate can be patterned with regions that alternate between hydrophilic and hydrophobic. For example, the substrate can be natively hydrophobic but patterned with small islands large enough to accommodate one or multiple drops with hydrophilic wettability. The wettability patterning can be accomplished with, for example, spatially-modulated light-based polymer grafting or flow patterning of polyelectrolyte layers. Once the droplets are in contact with the hydrophilic patch, they may wet spontaneously or they may be induced to wet, for instance if surfactants are present, by applying a small, transient or long-lived electric field. Once wetted to the substrate, the droplets can be maintained for periods of time.

A method to trap droplets, which utilizes interfacial tension, may be accomplished with patterned features. For example, wells can be fabricated into the substrate and sized and/or shaped such that droplets fit therein and, due to buoyancy or density differences with the carrier fluid, sit within the wells. The droplets can also be dispensed within a concave feature with a narrow opening, or between posts with narrow gaps. Such droplets may be held in place due to their interfacial tension and preference for remaining spherical.

Other kinds of electromagnetic traps can be generated using, for example, laser tweezers. Using an array of lasers directed at controlled locations on the substrate, droplets dispensed near the lasers may experience a force attracting or repelling them to or from the lasers, again generating a series of traps that can be used to localize the droplets. Magnetic droplets or particles can be affixed using magnetic or electromagnetic forces such as, for example, with ferrofluids, permanent magnets, paramagnetism, or electromagnetism generated by flowing electric current through an electrode patterned under the substrate.

Once droplets are dispensed to the substrate, it is possible to change the position of the droplets on the substrate. This can be accomplished, e.g., magnetically, by modulating the magnetic field, electrically or dielectrophoretically, by modulating electric fields, via electrowetting on dielectric, or by varying the position of optical traps with the lasers, among other forces. The carrier fluid, e.g., oil, surrounding the droplets can also be flowed so as to apply a shear to the droplets affixed to the substrate, causing them in some instances to move in the direction of the flow. If the droplets have a different density from the carrier fluid, buoyancy can also be used to move the droplets by altering the orientation of the substrate in a gravitational field.

In some embodiments, it may be desirable to dispense multiple droplets or discrete entities to a single location on the substrate array (e.g., to combine SD cells with a TM cells). This can be accomplished by, for example, dispensing a first droplet to the array and then dispensing a second droplet to the same position as the first droplet. In certain embodiments, such as when electric fields are used to trap the drops, the electric fields generated by the substrate are sufficient to induce the droplets to merge, thereby combining their contents. The contents of the droplets can be mixed via diffusion or convective flow in the droplets generated by, for example, convection of the carrier fluid over the surface of the droplet or motion of the droplet when the trap is moved. In other instances, droplets will merge spontaneously, such as when no surfactants are used. In other instances, merger can be induced via application of a laser or localized heating. Additional drops can be added to the same location to add, one, two, three, or more droplets to the same position. Using the droplet or sorting on demand techniques, the drops that are added and the sequence in which they are added can be controlled exactly. In another embodiment, a nozzle or capillary can be introduced into the affixed droplet to inject the desired reagent.

In certain applications, it is desirable to recover all or portions of the affixed droplets. This can be accomplished, for example, by bringing a nozzle close to the affixed droplet and drawing fluid into the nozzle, thereby drawing the droplet into the nozzle. Alternatively, the nozzle can be used to generate a localized flow of carrier fluid which can be used to dispel droplets from the surface by overcoming the affixing force. If the nozzle shape is designed appropriately and the fluid flow adequate, it is also possible to recover a portion of the droplet in a mechanism similar to microcapillary-based droplet generation. Alternatively, or in addition, droplets can be removed from the substrate by adding additional liquid to them to increase their buoyancy. Once the buoyant force is larger than the affixing force, the droplet will detach from the substrate and float away. If the droplets are heavier than the carrier, a similar result can be accomplished by inverting the substrate. In embodiments in which the traps can be selectively switched on and off, droplets can also be recovered by switching off the force and using buoyancy or flow to remove them from the substrate and recover them into a collection container.

The portions or complete droplets recovered with any of the methods described herein can then be dispensed into a secondary container by flowing them from the array into the container. For example, using the section method, individual droplets or droplet portions can be recovered from the droplet array and these portions flowed through a tube into a well on a well plate, where they are dispensed. This can be done one droplet at a time, dispensing each droplet into a separate well and thereby preserving the isolation of the droplets from one another. Once in the well, other operations can be performed on the droplet, such as propagating cells contained therein or performing biological reactions, such as lysing the cells and then sequencing the nucleic acid sequence therein.

In certain embodiments, the binding of SD cells to TM cells are measured. Multiple measurement modalities can be used, such as bright field, fluorescence, and absorbance techniques. Spectrographic techniques can also be applied, such as Raman spectroscopy, NMR, and mass spectrometry. Separation techniques can also be used, such as capillary electrophoresis by making contact with the droplets through, for example, a nozzle or capillary. By recovering all or portions of the droplets, the material can also be subjected to destructive or non-destructive techniques, such as mass-spectrometry and chemical analysis. Importantly, since the nozzle position is known during the material recovery process, the signals and information recovered from these and other assays can be traced back to specific droplets on the array, allowing time-resolved information to be combined with powerful molecular analysis techniques, such as sequencing of the material in the droplet portions.

In certain applications it is valuable to tag the contents of the printed droplets with a unique identifier. This allows for the contents of multiple droplets to be pooled together while keeping track of from which droplet each entity originated. One such example of this is nucleic acid tagging, or barcoding. In this approach, for example, single cells can be localized in the droplets, lysed, and their nucleic acids tagged with unique identifiers relating from which droplet, and thus, from which cell, each nucleic acid originated. This allows the nucleic acid that encodes the surface displayed protein (variable region) in the SD cells to be sequenced. The nucleic acids can then all be pooled and sequenced and the tags used to group them according to single droplets and cells.

In certain embodiments, the binding of SD cells to TM cells is analyzed by fluorescence. To analyze the fluorescence of a droplet, an excitation light is provided, e.g., in the form of a laser, and read the generated emission light. In some embodiments of the invention, this can be accomplished using a single optical fiber that serves both to funnel the excitation light into the device and also collects the emitted light in the reverse direction. A drawback of this approach, however, is that the optical properties that are ideal for excitation light guidance may not be the same as for emission light capture. For example, to excite a narrow beam, a fiber with a narrow tip is preferred, but to collect the largest number of emitted photons, a wide fiber with a large collecting cone angle is preferred. In these instances, multiple fibers can be used. For example, a narrow fiber can be used to provide a concentrated, excitation signal, while a wide fiber can collect the emitted fluorescent light.

In some embodiments, the methods, devices, and/or systems described herein can be used to detect nucleic acids, such as the variable region displayed on the surface of the SD cells. In certain embodiments, reagents necessary for amplification are added to the droplets, either by combining them with the sample droplets prior to dispensing, or by dispensing additional droplets to the positions of the sample containing droplets, wherein the additional droplets include the necessary reagents and a detection component, where the detection component signals the amplification. The droplets are then incubated under conditions suitable for amplification and monitored to read the detection component. This provides, for each droplet, a rate of change of the detection component which can be used to detect and/or quantitate the nucleic acids in the droplets.

In some embodiments, the methods, devices, and/or systems described herein can be used to sequence nucleic acid derived from single cells. For example, individual cells can be encapsulated in the droplets and dispensed to the substrate as described herein. The cells can then be lysed and subjected to molecular biological processing to amplify and/or tag their nucleic acids with barcodes. The material from all the droplets can then be pooled for all cells and sequenced and the barcodes used to sort the sequences according to single droplets or cells. These methods can be used, for example, to sequence the genomes or transcriptomes of single cells in a massively parallel format.

As described above, in certain embodiments, nucleic acid sequence assay components that employ barcoding for labelling individual mRNA molecules, and/or for labeling for cell/well source (e.g., if wells pooled before sequencing analysis), and/or for labeling particular affixed entities (e.g., if droplet from two or more affixed entities are pooled prior to sequencing) are employed. Examples of such barcoding methodologies and reagents are found in Pat. Pub. US2007/0020640, Pat. Pub. 2012/0010091, U.S. Pat. 8,835,358, U.S. Pat. 8,481,292, Qiu et al. (Plant. Physiol., 133, 475-481, 2003), Parameswaran et al. (Nucleic Acids Res. 2007 October; 35(19): e130), Craig et al. reference (Nat. Methods, 2008, October, 5(10):887-893), Bontoux et al. (Lab Chip, 2008, 8:443-450), Esumi et al. (Neuro. Res., 2008, 60:439-451), Hug et al., J. Theor., Biol., 2003, 221:615-624), Sutcliffe et al. (PNAS, 97(5):1976-1981; 2000), Hollas and Schuler (Lecture Notes in Computer Science Volume 2812, 2003, pp 55-62), and WO201420127; all of which are herein incorporated by reference in their entireties, including for reaction conditions and reagents related to barcoding and sequencing of nucleic acids.

In certain embodiments, the DropSeq method employing beads with primers attached to them are employed to sequence the variable regions from SD cells. An example of such a method is described in Macosko et al., Cell, 161(5):1202-1214 (see, e.g., FIG. 1), which is herein incorporated by reference in its entirety. In certain embodiments employing DropSeq, barcoded template switch oligos are bound to beads and oligo dT is supplied in solution along with RT PCR reagents. Reverse transcription (RT) can, for example, be performed as described in Perform RT PCR according to Kim et al., Anal Chem. 2018 Jan. 16; 90(2):1273-1279, herein incorporated by reference. In other embodiments, barcoded oligo-dT beads are provided, the cells are lysed, mRNAs is captured on the beads, the emulsion is broken, and the drop is re-emulsified to capture mRNA beads with barcoded TSO beads where the TSO can be released by UV. Solution phase TSO can then be used for performing RT-PCR. Primers specific to the variable regions displayed on the surface of the SD cells can be employed to amplify such variable regions prior to sequencing.

In certain embodiments, as shown in FIG. 1B, unique oligo drops are provided to the fixed entities, and allow a link between imaging and genomics. For example, the unique oligos can contain two part 8 mer barcodes linked to polyA or TSO followed by 8-mer barcodes. In this regard, if one employs 96 barcoded oligos, selecting any three can generate 142,880 combinations. It is known what combination of three oligos are printed at each well position to identify that particular well (e.g., so a variable region that binds a TM protein can be identified). These oligos will also be sequenced and so when one sees a particular 3-oligo combination in the sequencing readouts, one knows the fixed entity and the image for that fixed entity.

In certain embodiments, the barcode tagging and sequencing methods of WO2014201272 (“SCRB-seq” method, herein incorporated by reference) are employed. The necessary reagents for the SCRB-seq method (e.g., modified as necessary for small volumes) are added to the fixed entities, each containing a lysed cells. Briefly, the SCRB-seq method amplifies an initial mRNA sample from cells from a single fixed entity. Initial cDNA synthesis uses a first primer with: i) N6 for cell/well identification, ii) N10 for particular molecule identification, iii) a poly T stretch to bind mRNA, and iv) a region that creates a region where a second template-switching primer will hybridize. The second primer is a template switching primer with a poly G 3′ end, and 5′ end that has iso-bases. After cDNA amplification, the tagged cDNA single fixed entity samples are pooled. Then full-length cDNA synthesis occurs with two different primers, and full-length cDNA is purified. Next, a NEXTERA sequencing library is prepared using an i7 primer (adds one of 12 i7 tags to identify particular multi-well plates) and PSNEXTPTS to add P5 tag for NEXTERA sequencing (P7 tag added to other end for NEXTERA). The library is purified on a gel, and then NEXTERA sequencing occurs. As a non-liming example, with twelve i7 plate tags, and 384 cell/well-specific barcodes, this allows total of 4,608 single cell transciptomes to be done at once. This method allows for quantification of mRNA transcripts in single fixed entity.

In other embodiments, the barcode tagging and sequencing methods employ concepts from the Multi-seq method (see, e.g., [1]). For example, cells are incubated with anchor and co-anchor lipid modified oligonucleotides (LMO) and encapsulated in droplets. Individual barcodes in droplets can hybridize to exposed regions of the LMOs and these barcodes can be used instead of Drop-seq beads. Anchor-coanchor LMOs remain bound to individual cells at 4° C. but can freely equilibrate between cells in a droplet at 37° C. Thus, a specific LMO-barcode combination in each droplet can be used to link two cells in that droplet that can be tracked after emulsion breaking.

The composition and nature of the discrete entities, e.g., microdroplets, prepared and or utilized in connection with the disclosed methods may vary. In some embodiments, a surfactant may be used to stabilize the discrete entities, e.g., microdroplets. Accordingly, a microdroplet may involve a surfactant stabilized emulsion. Any surfactant that allows for the desired reactions to be performed in the discrete entities, e.g., microdroplets, may be used. In other aspects, a discrete entity, e.g., a microdroplet, is not stabilized by surfactants or particles. The surfactant used depends on a number of factors such as the oil and aqueous phases (or other suitable immiscible phases, e.g., any suitable hydrophobic and hydrophilic phases) used for the emulsions. For example, when using aqueous droplets in a fluorocarbon oil, the surfactant may have a hydrophilic block (PEG-PPO) and a hydrophobic fluorinated block (Krytox® FSH). If, however, the oil was switched to be a hydrocarbon oil, for example, the surfactant would instead be chosen so that it had a hydrophobic hydrocarbon block, like the surfactant ABIL EM90. In selecting a surfactant, desirable properties that may be considered in choosing the surfactant may include one or more of the following: (1) the surfactant has low viscosity; (2) the surfactant is immiscible with the polymer used to construct the device, and thus it doesn't swell the device; (3) biocompatibility; (4) the assay reagents are not soluble in the surfactant; (5) the surfactant exhibits favorable gas solubility, in that it allows gases to come in and out; (6) the surfactant has a boiling point higher than the temperature used for PCR (e.g., 95° C.); (7) the emulsion stability; (8) that the surfactant stabilizes drops of the desired size; (9) that the surfactant is soluble in the carrier phase and not in the droplet phase; (10) that the surfactant has limited fluorescence properties; and (11) that the surfactant remains soluble in the carrier phase over a range of temperatures. Other surfactants can also be employed, including ionic surfactants. Other additives can also be included in the oil to stabilize the discrete entities, e.g., microdroplets, including polymers that increase discrete entity, e.g., droplet, stability at temperatures above 35° C.

The discrete entities, e.g., microdroplets, described herein may be prepared as emulsions, e.g., as an aqueous phase fluid dispersed in an immiscible phase carrier fluid (e.g., a fluorocarbon oil or a hydrocarbon oil) or vice versa. The nature of the microfluidic channel (or a coating thereon), e.g., hydrophilic or hydrophobic, may be selected so as to be compatible with the type of emulsion being utilized at a particular point in a microfluidic work flow.

Emulsions may be generated using microfluidic devices. Microfluidic devices can form emulsions consisting of droplets that are uniform in size. The microdroplet generation process may be accomplished by pumping two immiscible fluids, such as oil and water, into a junction. The junction shape, fluid properties (viscosity, interfacial tension, etc.), and flow rates influence the properties of the microdroplets generated but, for a relatively wide range of properties, microdroplets of controlled, uniform size can be generated using methods like T-junctions and flow focusing. To vary microdroplet size, the flow rates of the immiscible liquids may be varied since, for T-junction and flow focus methodologies over a certain range of properties, microdroplet size depends on total flow rate and the ratio of the two fluid flow rates. To generate an emulsion with microfluidic methods, the two fluids are normally loaded into two inlet reservoirs (syringes, pressure tubes) and then pressurized as needed to generate the desired flow rates (using syringe pumps, pressure regulators, gravity, etc.). This pumps the fluids through the device at the desired flow rates, thus generating microdroplet of the desired size and rate.

As indicated above, lysing agents may be added to the discrete entities, e.g., droplets, containing a cell, under conditions in which the cell(s) may be caused to burst, thereby releasing their nucleic acid. The lysing agents may be added after the cells are encapsulated into discrete entities, e.g., microdroplets. Any suitable lysing agent may be employed, such as proteinase K or cytotoxins. In particular embodiments, cells may be co-encapsulated in drops with lysis buffer containing detergents such as Triton X100 and/or proteinase K. The specific conditions in which the cell(s) may be caused to burst will vary depending on the specific lysing agent used. For example, if proteinase K is incorporated as a lysing agent, the discrete entities, e.g., droplets, may be heated to about 37-60° C. for about 20 min to lyse the cells and to allow the proteinase K to digest cellular proteins, after which they may be heated to about 95° C. for about 5-10 min to deactivate the proteinase K. In certain aspects, cell lysis may also, or instead, rely on techniques that do not involve addition of lysing agent. For example, lysis may be achieved by mechanical techniques that may employ various geometric features to effect piercing, shearing, abrading, etc. of cells. Other types of mechanical breakage such as acoustic techniques may also be used. Further, thermal energy can also be used to lyse cells.

In some embodiments, systems and/or devices are provided which include one or more discrete entity printers. Discrete entity printers may include one or more microfluidic device, such as a microfluidic device including one or more discrete entity makers (e.g., droplet makers) configured to generate discrete entities (e.g., droplets), and/or one or more flow channels. In some aspects, the one or more flow channels are operably connected, e.g., fluidically connected, to the one or more droplet makers and/or are configured to receive one or more droplets therefrom. By “operably connected” and “operably coupled”, as used herein, is meant connected in a specific way (e.g., in a manner allowing fluid, e.g., water, to move and/or electric power to be transmitted) that allows a disclosed system or device and its various components to operate effectively in the manner described herein.

In certain embodiments, the microfluidic devices include one or more delivery orifice, such as a delivery orifice fluidically connected to one or more flow channels. In some embodiments, delivery orifices include an opening, e.g., a circular or oblong opening, through which one or more discrete entities may pass. In some embodiments, openings of delivery orifices are defined by a rim of a device or a portion thereof, e.g., a nozzle that can be precisely positioned. Delivery orifices, as included in the subject embodiments, may have any of the same dimensions, e.g., a cross-sectional dimension, as the flow channels described herein, or may have different dimensions.

A delivery orifice of a microfluidic nozzle as described herein, will generally have dimensions that are similar to the size of the droplets to be delivered therethrough. Accordingly, in some embodiments, a delivery orifice as described herein has a diameter of from about 1 μm to about 1000 μm, inclusive, e.g., from about 10 μm to about 300 μm, inclusive. In some embodiments, a delivery orifice as described herein has a diameter of from about 1 μm to about 10 μm, from about 10 μm to about 100 μm, from about 100 μm to about 500 μm, or from about 500 μm to about 1000 μm, inclusive. The nozzle can be molded as part of a microfluidic sorter as described herein, or can be a separate part that is mated with a microfluidic sorter as described herein. Suitable materials for the nozzle may include, e.g., polymeric tubing, small bore hypodermic tubing, and modified glass capillaries.

In certain embodiments, one or more automated system are integrated with the delivery orifice, wherein the automated system (a) selectively positions, e.g., positions by moving one or more distance on the order of magnitude of a discrete entity, the delivery orifice in proximity to a substrate or a portion thereof during operation and/or (b) selectively positions, e.g., positions by moving one or more distance on the order of magnitude of a discrete entity, the substrate or portion thereof in proximity to the delivery orifice during operation, such that a discrete entity, e.g., a droplet, can be ejected from the delivery orifice and/or deposited on the substrate. In some embodiments, automated systems are electronic and/or include one or more control unit for controlling automation, such as a control unit including a central processing unit.

As noted above, droplet printers may include one or more flow channels, e.g., flow channels which discrete entities may pass into, out of, and/or through. In certain embodiments, flow channels are one or more “micro” channel. Such channels may have at least one cross-sectional dimension on the order of a millimeter or smaller (e.g., less than or equal to about 1 millimeter). For certain applications, this dimension may be adjusted. In some embodiments at least one cross-sectional dimension is about 500 micrometers or less. In some embodiments, the cross-sectional dimension is about 100 micrometers or less, or about 10 micrometers or less, and sometimes about 1 micrometer or less. A cross-sectional dimension is one that is generally perpendicular to the direction of centerline flow, although it should be understood that when encountering flow through elbows or other features that tend to change flow direction, the cross-sectional dimension in play need not be strictly perpendicular to flow. It should also be understood that in some embodiments, a micro-channel may have two or more cross-sectional dimensions such as the height and width of a rectangular cross-section or the major and minor axes of an elliptical cross-section. Either of these dimensions may be compared against sizes presented here. Note that micro-channels employed in this disclosure may have two dimensions that are grossly disproportionate—e.g., a rectangular cross-section having a height of about 100-200 micrometers and a width on the order or a centimeter or more. Of course, certain devices may employ channels in which the two or more axes are very similar or even identical in size (e.g., channels having a square or circular cross-section).

In certain embodiments, microfluidic devices are fabricated using microfabrication technology. Such technology may be employed to fabricate integrated circuits (ICs), microelectromechanical devices (MEMS), display devices, and the like. Among the types of microfabrication processes that can be employed to produce small dimension patterns in microfluidic device fabrication are photolithography (including X-ray lithography, e-beam lithography, etc.), self-aligned deposition and etching technologies, anisotropic deposition and etching processes, self-assembling mask formation (e.g., forming layers of hydrophobic-hydrophilic copolymers), etc.

When referring to a microfluidic device it is sometimes intended to represent a single entity in which one or more channels, reservoirs, stations, etc. share a continuous substrate, which may or may not be monolithic. Aspects of microfluidic devices include the presence of one or more fluid flow paths, e.g., channels, having dimensions as discussed herein. A microfluidics system may include one or more microfluidic devices and associated fluidic connections, electrical connections, control/logic features, etc. In certain embodiments, the microfluidic systems include one or more discrete entity printer, e.g., one or more droplet printer, and/or a substrate or portion thereof, e.g., a substrate surface, for receiving one or more discrete entities, e.g., droplets deposited thereon by, for example, a delivery orifice of a discrete entity printer, e.g., a droplet printer. Systems may also include one or more of: (a) a temperature control module for controlling the temperature of one or more portions of the subject devices and/or discrete entities therein and which is operably connected to the discrete entity printer, e.g., a droplet printer, (b) a detection means, i.e., a detector, e.g., an optical imager, operably connected to the discrete entity printer, e.g., a droplet printer, (c) an incubator, e.g., a cell incubator, operably connected to the discrete entity printer, e.g., a droplet printer, and (d) a sequencer operably connected to the discrete entity printer, e.g., a droplet printer. The subject systems may also include one or more conveyor configured to move, e.g., convey, a substrate from a first discrete entity, e.g., droplet, receiving position to one or more of (a)-(d). The microfluidic devices and systems may include one or more sorter for sorting discrete entities, e.g., droplets, into one or more flow channels. Such a sorter may sort and distribute discrete entities, e.g., droplets, based on one or more characteristics of the discrete entities including composition, size, shape, buoyancy, or other characteristics. In certain embodiments, the microfluidic systems and devices include one or more detection components i.e., a detector, e.g., an optical imager, configured for detecting the presence of one or more discrete entities, e.g., droplets, or one or more characteristics thereof, including their composition. In some embodiments, detection components are configured to recognize one or more components of one or more discrete entities, e.g., discrete entities, in one or more flow channel. In some embodiments, the devices and systems include one or more temperature and/or pressure control module. Such a module may be capable of modulating temperature and/or pressure of a carrier fluid in one or more flow channels of a device. In certain embodiments, a temperature control module may be one or more thermal cycler.

In certain embodiments, the substrates used in microfluidic devices and/or systems are the supports in which the necessary elements for fluid transport are provided. The basic structure of a substrate may be, for example, monolithic, laminated, or otherwise sectioned. Substrates may include one or more flow channels, such as microchannels serving as conduits for molecular libraries and/or reagents. They may also include input ports, output ports, and/or features to assist in flow control. In certain embodiments, the substrate choice may be dependent on the application and design of the device. Substrate materials may be chosen for their compatibility with a variety of operating conditions. Limitations in microfabrication processes for a given material are also relevant considerations in choosing a suitable substrate. Useful substrate materials which may be employed with the subject disclosure include, e.g., glass, polymers, silicon, metal, ceramics, and/or combinations thereof.

### REFERENCES

[1] Rodgers et al., “Glimmers in illuminating the druggable genome,” Nat. Rev. Drug Discov., vol. 17, p. 301, January 2018.
[2] Xiao et al., “High Throughput Screening for Orphan and Liganded GPCRs,” Combinatorial Chemistry & High Throughput Screening, vol. 11, no. 3. pp. 195-215, 2008.
[3] Nieto Gutierrez and P. H. McDonald, “GPCRs: Emerging anti-cancer drug targets,” Cell. Signal., vol. 41, no. September, pp. 65-74, 2018.
[4] B. G. Yadav, “Trends in Antibody Generation Techniques—the Fully Synthetic Human Combinatorial Antibody Library (HuCAL®) powerful,” 2015.
[5] C. McMahon et al., “Yeast surface display platform for rapid discovery of conformationally selective nanobodies,” Nat. Struct. Mol. Biol., vol. 25, no. 3, pp. 289-296, 2018.
[6] D. Huang and E. V. Shusta, “A yeast platform for the production of single-chain antibody-green fluorescent protein fusions,” Appl. Environ. Microbiol., vol. 72, no. 12, pp. 7748-7759, 2006.
[7] R. H. Cole et al., “Printed droplet microfluidics for on demand dispensing of picoliter droplets and cells,” Proc. Natl. Acad. Sci., July 2017.
[8] C. A. Siltanen et al., “An Oil-Free Picodrop Bioassay Platform for Synthetic Biology,” Sci. Rep., vol. 8, no. 1, pp. 1-7, 2018.
[9] E. Z. Z. Macosko et al., “Highly Parallel Genome-wide Expression Profiling of Individual Cells Using Nanoliter Droplets,” Cell, vol. 161, no. 5, pp. 1202-1214, 2015.
[1] C. S. McGinnis et al., “MULTI-seq: sample multiplexing for single-cell RNA sequencing using lipid-tagged indices,” Nat. Methods, vol. 16, no. 7, pp. 619-626, 2019.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method comprising:
a) flowing a plurality of discrete entities through a micro-fluidic device in a carrier fluid, wherein said discrete entities are insoluble and/or immiscible in the carrier fluid, and wherein each of said discrete entities comprises: i) one, and no more than one, transmembrane protein expressing cell (TM cell), or ii) a plurality of TM cells where each of said TM cells expresses the same transmembrane protein;
b) directing said carrier fluid and said plurality of discrete entities through a delivery orifice to a substrate such that each of said discrete entities merges with one of a plurality of affixed entities present on said substrate thereby generating a plurality of merged affixed entities,
wherein each of said affixed entities comprises a plurality of clonal surface display cells (SD cells), wherein each of said clonal SD cells comprises:
i) an outer surface displaying a polypeptide, wherein said polypeptide comprises at least one heterologous antibody variable region that is unique among the heterologous variable regions present in said plurality of affixed entities,
ii) a nucleic acid sequence encoding said polypeptide, and
iii) a detectable protein; and
c) detecting directly or indirectly, in each of said plurality of merged affixed entities, whether said clonal SD cells bind to said TM cell or to said plurality of TM cells.

2. The method of claim 1, further comprising: identifying at least one merged affixed entity where said multiple SD cells bound to said TM cell or plurality of TM cells, and adding a composition to said at least one merged affixed entity, wherein said composition comprises barcoded nucleic acid sequences that bind to said nucleic acid sequence encoding said polypeptide.

3. The method of claim 2, further comprising: i) sequencing said nucleic acid sequence encoding said polypeptide to determine the sequence encoding said heterologous antibody variable region.

4. The method of claim 1, wherein said detecting comprises detecting the position of said SD cells via said detectable protein.

5. The method of claim 1, wherein said detectable protein comprises a fluorescent protein.

6. The method of claim 1, wherein said plurality of first discrete entities comprises at least 1000 first discrete entities or at least 10,000 first discrete entities.

7. The method of claim 1, wherein said plurality of SD cells comprises at least 1000 SD cells or at least 10,000 SD cells.

8. The method of claim 1, wherein said substrate is configured to move to different positions under the delivery orifice.

9. The method of claim 1, wherein said plurality of affixed entities are affixed to said substrate via a force, wherein said force is selected from: gravitational force, electrical force, magnetic force, and combinations thereof.

10. The method of claim 1, wherein said plurality of affixed entities are affixed to said substrate via an electrical force.

11. The method of claim 10, wherein said electrical force is a dielectrophoretic force.

12. The method of claim 1, wherein said discrete entities are droplets.

13. The method of claim 12, wherein said droplets comprise an aqueous fluid which is immiscible in said carrier fluid.

14. The method of claim 13, wherein said substrate comprises on a first surface a layer of fluid which is miscible with said carrier fluid and immiscible with said aqueous fluid, and wherein said first discrete entities are affixed to said first surface of said substrate following introduction into said layer of fluid on said first surface of said substrate.

15. The method of claim 12, wherein the carrier fluid is an aqueous fluid and the droplets comprise a fluid which is immiscible with the carrier fluid.

16. The method of claim 15, wherein the substrate comprises on a first surface a layer of aqueous fluid which is miscible with the carrier fluid and immiscible with the fluid comprised by the droplets, and wherein the first discrete entities are affixed to the first surface of the substrate following introduction into the layer of aqueous fluid on the first surface of the substrate.

17. The method of claim 1, wherein said first discrete entities are affixed to the substrate via interfacial tension.

18. The method of claim 1, wherein said polypeptide comprises at least two heterologous antibody variable regions.

19. A system comprising:
a) a substrate comprising a first surface;
b) a layer of fluid covering at least part of said first surface; and
c) a plurality of affixed entities,
wherein each of said affixed entities are independently affixed to said substrate under said layer of fluid,
wherein said plurality of affixed entities are insoluble and/or immiscible in said layer of fluid,
wherein each of said plurality of affixed entities comprises:
i) one, and no more than one, TM cell expressing at least one transmembrane protein, or a plurality of TM cells where each of said TM cells expresses the same transmembrane protein, and
ii) multiple identical surface display cells (SD cells), wherein each of said SD cells comprises:
A) an outer surface displaying a polypeptide, wherein said polypeptide comprises at least one heterologous antibody variable region that is unique among the heterologous variable regions present in said plurality of affixed entities,
B) a nucleic acid sequence encoding said polypeptide, and
C) a detectable protein.
